(19) United States
(12) Patent Application Publication
(10) Pub. No.: US 2011/0038811 A1
(43) Pub. Date: Feb. 17, 2011

(54) COMPOSITION

(76) Inventors: Abid Nadim Khan-Lodhi, Bebington (GB); Ezat Khoshdel, Wirral (GB); Jasmin Patricia Menzel, Coventry (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/920,470

(22) PCT Filed: Mar. 5, 2009
(86) PCT No.: PCT/EP09/52597

§ 371 Date: Oct. 29, 2010

(30) Foreign Application Priority Data
Mar. 14, 2008 (EP) 08152751.7

(51) Int. Cl. C07H 1/00; A61K 8/73; A61Q 15/00; A61Q 11/00; C11D 3/37; C09D 11/14; C09D 105/16; C09K 3/00

(52) U.S. Cl. 424/49; 536/123.1; 424/70.13; 424/65; 510/276; 510/130; 106/31.13; 106/205.01; 252/182.13

(57) ABSTRACT

Cationic-modified polymer cross-linked with a cross-linker, characterized in that the cross-linker is selected from fatty acids having at least 12 carbon atoms and anionic surfactants having at least 10 carbon atoms. A personal care composition comprising such a polymer and a method for cross-linking a polymer by reacting with a cross-linker having at least two anionic groups.

The present invention provides a cross-linked polymer. The present invention also provides a composition comprising a cross-linked polymer and a method for making a cross-linked polymer.

EP-A-1 490 408 (Lamberti) discloses the use of glyoxal to cross-link cationically modified guar.

Despite the prior art there remains a need for cross-linked cationic-modified polymers which do not use formaldehyde, boron or glyoxal.

Accordingly, there is provided a cationic-modified polymer cross-linked with a cross-linker, characterized in that the cross-linker is selected from fatty acids having at least 12 carbon atoms and anionic surfactants having at least 10 carbon atoms.

Preferably, the polymer is selected from polysaccharides, polyvinyl alcohols, polyacrylates, polymethacrylates, polystyrenes and polyurethanes. More preferably, it is a polysaccharide, for example carboxymethyl cellulose, carboxymethyl starch and carboxymethyl hydroxyethylcellulose. Most preferably it is a guar or a derivative thereof.

Preferably, the fatty acid comprises a straight chain alkyl group. This assists in providing an effective cross-linker.

A most preferred fatty acid is lauric acid.

Preferably, the surfactant comprises a straight chain alkyl group. This also assists in providing an effective cross-linker.

Preferably, the surfactant comprises an anionic group. Preferably, the anionic group is selected from carboxylates, sulphonates, sulphates and phosphonates.

Most preferred surfactants include sodium decyl sulphate, sodium dodecyl sulphate, sodium tetradecyl sulphate, sodium lauryl ether sulphate comprising one or two ethoxylated groups.

The cross-linking is reversed by addition of salt. Such salt addition can be incorporated into the formulating of a composition or part of a separate process. Suitable formulations which may comprise such cationic-modified polymer include personal care formulation such a hair care compositions, personal wash compositions, deodorant compositions, laundry and oral care compositions.

Alternative compositions include paints, water treatment compositions and inkjet print.

Suitable salts include sodium, potassium, cesium, ammonium chloride, bromide, iodide, sulphate, sulphonate and phosphate.

It should be appreciated that the cross-linked polymer will not be cross-linked in the presence of suitable uncrosslinking electrolytes.

For the avoidance of doubt the invention relates to a crosslinked polymer composition in which at least 95% of the polymer is crosslinked. Alternatively, the composition comprises less than 0.1%, preferably less than 0.05% by weight uncrosslinker.

More preferably, the composition consists essentially of the crosslinked polymer and the crosslinker.

In a second aspect there is provided a method for preparing a cross-linked cationic-modified polymer.

Such a method comprises reacting a cross-linker as herein described in an aqueous polymer solution.

In a third aspect the invention provides a method for uncross-linking a cross-linked polymer according to the first aspect of the invention by reducing the pH of a composition comprising the cross-linked polymer.

In a fourth aspect the invention provides a method for uncross-linking a cross-linked polymer according to the first aspect by adding electrolyte to a composition comprising the cross-linked polymer. Suitable electrolytes include sodium chloride.

In a fifth aspect the invention provides a method for uncross-linking a cross-linked polymer according to the first aspect by adding cyclodextrin to a composition comprising the cross-linked polymer.

With regard to the third, fourth and fifth aspects of the invention, sufficient uncross-linker is added to resolubilise the polymer.

In a sixth aspect the invention provides a composition obtainable by a method according to any of the fourth to sixth aspects.

### EXAMPLE

All concentrations are referred to as weight percent.

Preparation of the Solutions

Quaternisation of Guar:

The guar gum is extracted with methanol to remove methanol soluble oils. The dried guar gum is dissolved in water or water/isopropanol mixtures (50:50), then heated to 40° C. The caustic solution is added to the solution or slurry and stirred for 15 minutes. The quaternising agent, 4-chloro-2-butenyl trimethylammoium chloride is added to the mixture and the etherification reaction is conducted at 40° C. for a period of 5 hours. The reaction mixture is cooled to room temperature and the crosslinkers, bis- or polyacids, e.g. polyacrylate, or SDS was added. The resultant precipitate was filtered wand air dried, or freeze dried.

The full details of the crosslinking agents are given below.

A 1% solution of Jaguar C17 was prepared by the following procedure:

After dissolving 1.6 g Glydant in 394.4 g water, 4 g Jaguar flower was added slowly while stirring with an electric stirrer. All solutions were stirred until the Jaguar flower was dissolved completely and swollen. The solutions became viscous in that time. 3 equal solutions of 400 g each were prepared.

A 2% solution was prepared of every compound used for a crosslinking experiment. Carboxylic acids with defined basicity were dissolved in an equibasic amount of 0.5 M sodium hydroxide solution and diluted with water until a concentration of 2% of the crosslinker was reached. Surfactants were only dissolved in water. Polyelectrolytes were dissolved in an approximated amount of 0.5 M sodium hydroxide solution and diluted with water to a 2% solution.

Table 4.1 gives the exact amounts for every compound.

TABLE 4.1
Exact amounts for the preparation of 2%
solutions of potential crosslinkers

| | m (compound) | m (0.5 M NaOH) | m (H2O) |
|---|---|---|---|
| | [g] | [g] | [g] |
| Compound | | | |
| Lauric acid | 1.00 | 10.00 | 39.00 |
| Linoleic acid | 0.25 | 1.78 | 10.47 |
| Surfactants | | | |
| Sodium decyl sulfate | 1.00 | 0.00 | 49.00 |
| Sodium dodecyl sulfate | 1.00 | 0.00 | 49.00 |
| Sodium tetradecyl sulfate | 1.00 | 0.00 | 49.00 |
| SLES (1EO) (70% aq) | 1.00 | 0.00 | 34.00 |
| SLES (2EO) (70% aq) | 1.00 | 0.00 | 34.00 |

A saturated sodium chloride solution was prepared by mixing 40 g NaCl with 100 g distilled water. The mixture was stirred for 6 hours.

2% solutions of the cyclodextrins were prepared by dissolving 1 g of β-cyclodextrin and (2-hydroxypropyl)-β-cyclodextrin respectively in 49 g distilled water.

Crosslinking

For each crosslink experiment a 25 g sample of the 1% Jaguar solution was prepared. 3 ml of the 2% solution of the different compounds were mixed with these samples. After measuring the pH of the mixtures, 0.5 M sodium hydroxide solution was added (if necessary) until a pH of 8-9 was reached. Tables 4.2-4.7 show the measured pH values and the observations.

TABLE 4.2
pH values and observations after adding the fatty
acids and adjusting the pH

| Fatty acids | pH | Observation |
|---|---|---|
| Lauric acid | 8.6 | thick gel precipitates, no phase |
| | | separation |
| Linoleic acid | 8.4 | thick lump of gel |

TABLE 4.3
pH values and observations after adding the
surfactants and adjusting the pH

| Surfactants | pH | Observation |
|---|---|---|
| Sodium decyl sulfate | 8.6 | big lump of gel; 2 clearly separated |
| | | phases |
| Sodium dodecyl | 8.4 | thick slime formed; 2 phases: |
| sulfate | | water + precipitate |
| Sodium tetradecyl | 8.4 | thick gel precipitates, one phase |
| sulfate | | |
| SLES (1EO) | 8.4 | fluffy precipitate, no stable gel |
| SLES (2EO) | 8.6 | fluffy precipitate, no stable gel |

Breaking the Crosslinks

Three different methods were tried to break the crosslinks between the Jaguar molecules.

In a first experiment the pH in the crosslinked solutions was decreased to around 5.5 by adding citric acid (50% aq). This or similar pH values are mostly used in shampoo and conditioner formulations.

When the acidification was not successful in breaking the crosslinks, a saturated sodium chloride solution was added dropwise until the precipitate disappeared or when it did not seem reasonably to add any more.

In a third experiment with new samples (also prepared according to 4.2) the pH was set to around 5.5 and then a 2% solution of (2-hydroxypropyl)-β-cyclodextrin and β-cyclodextrin respectively was added.

Table 4.8 shows the values and observations of these methods.

TABLE 4.5
Values and observations of the methods for
decrosslinking

| | Uncrosslinking | | |
|---|---|---|---|
| Cross-linker | method (amount) | pH | observations |
| Lauric acid | citric acid | 5.2 | still very viscous and |
| | | | hazy, more |
| | | | homogeneous |
| | NaCl | 5.2 | No precipitate left, |
| | | | still hazy |
| | 1* | | |
| | 2* | | |
| sodium decyl | citric acid | 6.1 | still very viscous and |
| sulfate | | | hazy |
| | NaCl | | No further change |
| | 1* | | |
| | 2* | | |
| sodium dodecyl | citric acid | 5.5 | Still precipitated |
| sulfate | NaCl (6 ml) | 5.5 | Solution gets clearer, |
| | | | foam is built when |
| | | | shaking, still hazy, |
| | | | but precipitate gone |
| | 1* | | |
| | 2* | | |
| sodium tetradecyl | citric acid | 5.8 | precipitate, thick gel |
| sulfate | NaCl | 5.8 | No precipitate left, |
| | | | still hazy |
| | 1* | | |
| | 2* | | |
| SLES (1EO) | citric acid | 5.5 | Still precipitated |
| | NaCl (6 ml) | 5.5 | Solution gets clearer, |
| | | | foam is built when |
| | | | shaking, still hazy, |
| | | | but precipitate gone |
| | 1* | | |
| | 2* | | |
| SLES (2EO) | citric acid | 5.5 | Still precipitated |
| | NaCl (6 ml) | 5.5 | Solution gets clearer, |
| | | | foam is built when |
| | | | shaking, still hazy, |
| | | | but precipitate gone |
| | 1* | | |
| | 2* | | |

1*: β-cyclodextrin;
2*: (2-hydroxypropy1)-β-cyclodextrin.

1. Cationic-modified polymer cross-linked with a cross-linker, characterized in that the cross-linker is selected from fatty acids having at least 12 carbon atoms and anionic surfactants having at least 10 carbon atoms.

2. Cross-linked polymer according to claim 1 wherein the fatty acid comprises a straight chain alkyl group.

3. Cross-linked polymer according to claim 1 wherein the surfactant comprises an alkyl group and an anionic moiety selected from sulphate, sulphonate and carboxylate.

4. Cross-linked polymer according to claim 3 wherein the alkyl group is a straight chain alkyl group.

5. A personal care composition comprising a cross-linked polymer according to claim 1.

6. A hair treatment composition comprising a cross-linked polymer according to claim 1.

7. A method for cross-linking a cationic-modified polymer by reacting said polymer in water and in the presence of a cross-linker selected from fatty acids having at least 12 carbon atoms and anionic surfactants having at least 10 carbon atoms.

8. A method for uncross-linking a cross-linked polymer according to claim 1 by reducing the pH of a composition comprising the cross-linked polymer.

9. A method for uncross-linking a cross-linked polymer according to claim 1 by adding electrolyte to a composition comprising the cross-linked polymer.

10. A method for uncross-linking a cross-linked polymer according to claim 1 by adding cyclodextrin to a composition comprising the cross-linked polymer.

11. A composition obtainable by a method according to claim 1.
